(19) United States
(12) Patent Application Publication
(10) Pub. No.: US 2009/0082730 A1
(43) Pub. Date: Mar. 26, 2009

(54) NEEDLE INJECTION ASSEMBLY

(76) Inventors: Li Nguyen, Westminster, CA (US); Athena Phan, Westminster, CA (US); Tiffany Nguyen, Westminster, CA (US)

Correspondence Address: KLEIN, O'NEILL & SINGH, LLP, 43 CORPORATE PARK, SUITE 204, IRVINE, CA, 92606, US

(21) Appl. No.: 12/237,315

(22) Filed: Sep. 24, 2008

Related U.S. Application Data
(60) Provisional application No. 60/974,536, filed on Sep. 24, 2007.

(51) Int. Cl. A61M 5/20

(52) U.S. Cl. 604/151

(57) ABSTRACT

An injection device is provided to deposit fluids or medications under the skin. The skin is first drawn up into a cavity of an injection head using a vacuum source. A telescoping housing is then activated to move an entire syringe so that a needle on the syringe penetrates the skin that has been pulled up into the injection head. In one embodiment, the syringe barrel is then retraced away from the skin to deposit fluids under the skin. In another embodiment, the syringe barrel is held stationary while the plunger rod is moved to deposit fluids under the skin.

### CROSS-REFERENCE TO RELATED APPLICATION(S)

This is a regular utility application of provisional application Ser. No. 60/974,536, filed Sep. 24, 2007, the contents of which are expressly incorporated herein by reference as if set forth in full.

Devices for administering an injection are generally discussed herein with particular discussions extended to devices for stimulating a cavity below the skin and administering a dosage in the stimulated cavity. Method for using and for making said devices are also discussed. The dosage can be medication for health and/or cosmetic treatments.

### BACKGROUND

There are a number of means for delivering medications to a patient to treat diseases and illnesses. Historically when using a syringe to deliver medications, a subject will generally be poked by a needle, either in the arm, leg, or buttock, and the medications delivered to the injection site through the lumen in the needle. The injection can vary from one caregiver to another as the location and depth of an injection can vary from one caregiver to another, which, for the most part, is not critical.

More recently, syringes are used to deliver medications for treating wrinkles, i.e., for cosmetic reasons. In these instances, a caregiver may randomly inject winkle areas of the face at varying depths to deliver neurotoxin, such as BOTOX®, or injectable fillers, such as ZYDERM®, ZYPLAST®, RESTYLANE®, RADIANCE®, and ARTFILL®, just to name a few. As medications can be injected to wrinkle areas at random depths, their effectiveness and efficiency are less than optimal.

Accordingly, there is a need for a device that ensures a more consistent delivery of medication and method of using same. Such a device not only can be used for treating wrinkles but can also be used for non-cosmetic treatment, such as for delivering anesthesia and slow release birth control medications.

This application may be related to and expressly incorporates by reference PCT Application No. PCT/US2005/047082, Publication No. WO 2006/069380, filed Jun. 29, 2006, entitled “DEVICE FOR ADMINISTERING AN INJECTION AND METHOD OF USING SAME”.

### SUMMARY

Aspects of the present invention include an injection device for performing an injection comprising a pump end for pushing a needle to perform an injection; an injection head coupled to the pump end, said injection head comprising a housing defining a cavity and having a perimeter defining an opening along a first plane; a vacuum source coupled to the injection head for providing a vacuum in the cavity of the housing; and wherein the needle is moveable along a second plane, which is non-perpendicular to the first plane.

In a further aspect of the present invention, an injection device for performing an injection is provided comprising a pump end for pushing a needle mounted on a syringe to perform an injection; an injection head connected to the pump end: said injection head comprising a nozzle and an opening defining a plane for placing against the skin; a telescoping end having the syringe placed therein; said telescoping end comprising a proximal housing and a distal housing; and wherein the proximal housing and the distal housing are configured to telescopically move relative to one another when a vacuum source is applied,

In still yet another aspect of the present invention, there is provided a injection

device for performing an injection comprising a pump end for pushing a needle mounted on a syringe to perform an injection; an injection head connected to the pump end; said injection head comprising a nozzle and an opening defining a plane for placing against the skin; a telescoping end having the syringe placed therein; said telescoping end comprising a proximal housing and a distal housing; a manifold connected to the telescoping end comprising at least one valve having a valve handle.

### BRIEF DESCRIPTION OF THE DRAWINGS

FIG. 1 is a transparent perspective view of a needle device provided in accordance with aspects of the present invention. In practice, the needle device may be opaque or non-transparent.

FIG. 2 is a transparent perspective view of an injection head provided, in accordance with aspects of the present invention.

FIG. 3 is a transparent perspective view of the injection device of FIG. 1 shown without the injection head of FIG. 2.

FIG. 4 is a transparent perspective view of the injection device of FIG. 1 following application of vacuum to a first interior space of the pump end of the injection device of FIG. 1.

FIG. 5 is a transparent perspective view of the injection device of FIG. 1 following application of vacuum to a second interior space of the pump end of the injection device of FIG. 1.

FIG. 6 is a transparent and cross-sectional view of the injection head placed against the skin.

FIG. 7 is a transparent and cross-sectional view of the injection head placed against the skin with a vacuum supplied to the injection head to draw up the skin into the interior cavity of the injection head.

FIG. 8 is a transparent and cross-sectional view of the injection head placed against the skin with a vacuum supplied to the injection head to draw up the skin into the interior cavity of the injection head and the injection device activated to puncture the skin with a needle.

FIG. 9 is a transparent and cross-sectional view of FIG. 8 with the needle retracting away from the skin to deposit fluids.

### DETAILED DESCRIPTION

The detailed description set forth below in connection with the appended drawings is intended as a description of the presently preferred injection device embodiments provided in accordance with aspects of the present invention and is not intended to represent the only forms in which the present invention may be constructed or utilized. The description sets forth the features and the steps for constructing and using the injection devices of the present invention in connection with the illustrated embodiments. It is to be understood, however, that the same or equivalent functions and structures may be accomplished by different embodiments that arc also intended to be encompassed within the spirit and scope of the invention. As denoted elsewhere herein, like element numbers are intended to indicate like or similar elements or features.

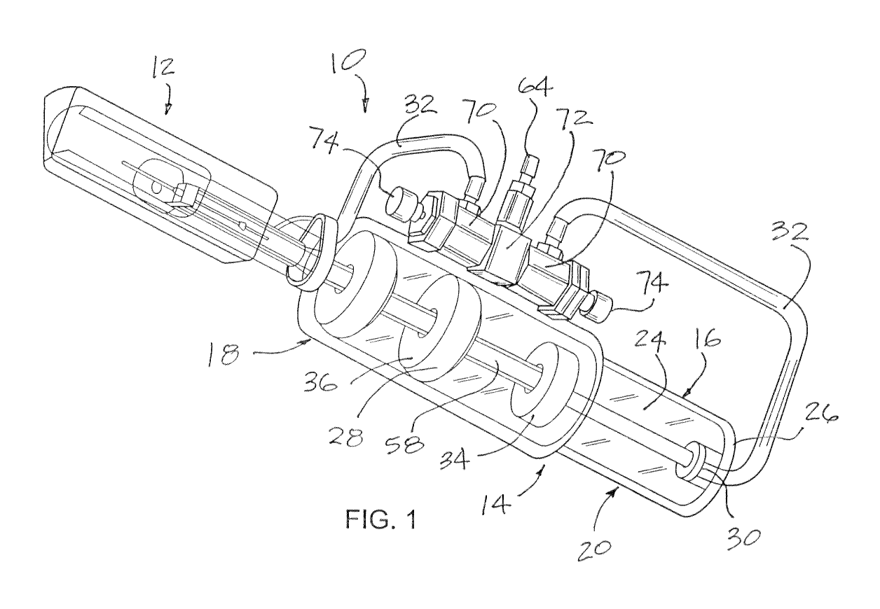

Referring now to FIG. 1, a perspective transparent view of an injectable device 10 provided in accordance with aspects of the present invention is shown. Broadly speaking, the injection device comprises an injection head 12 and a pump unit 14. In one embodiment, the pump unit 14 comprises a telescoping device 16, which comprises a distal housing 18 for receiving a proximal housing 20, and a connection device 22, which is configured to connect to a vacuum source for drawing a vacuum inside the cavity or interior space 24 defined by the distal and proximal housings 18, 20, as further discussed below. The use of a vacuum source to propel a piston for performing an injection has been disclosed in Publication No. WO 2006/069380, which has been incorporated herein by reference. In one embodiment, similar vacuum source may be used with the device 10 of the present invention.

In one embodiment, the proximal housing 20 comprises a generally cylindrical housing comprising a closed proximal end 26 comprising an end wall and an open distal end 28. To provide a communication link between the proximal housing 20 and a vacuum source, a hole or opening 30 is provided on the closed proximal end wall 26, which is shown connected to a tubing 32. In another less preferred embodiment, the opening for the communication link is provided elsewhere on the proximal housing 20 at a location proximal of the first barrel plunger 34. The open distal end 28 is shown closed by a second barrel plunger 36.

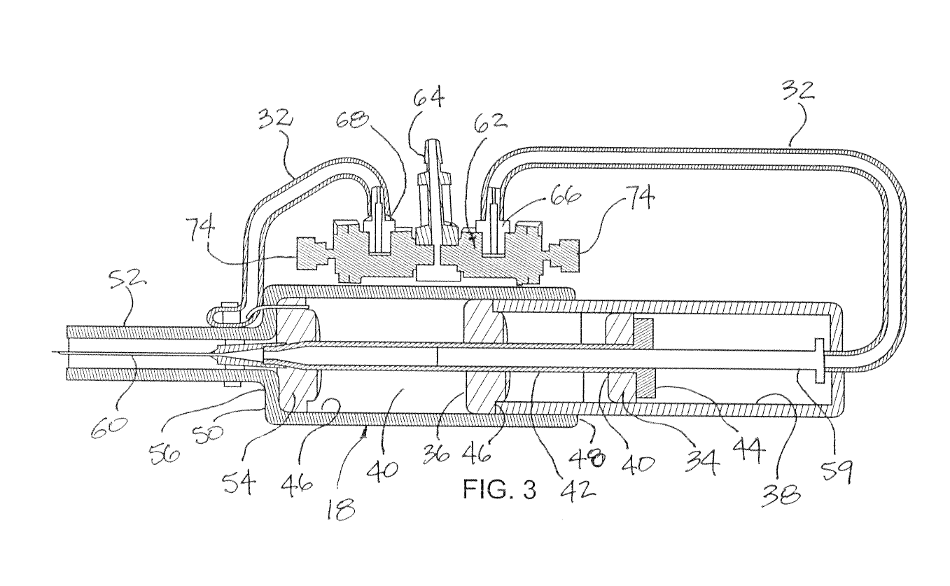

With reference now to FIG. 3 in addition to FIG. 1, the first barrel plunger 34

is configured to be in dynamic sealing arrangement with the interior wall surface 38 of the proximal housing 20. The first barrel plunger 34, and other plungers discussed elsewhere herein, is similar to a conventional syringe plunger for providing a seal between a piston rod and a syringe barrel with one exception. The first barrel plunger 34 incorporates a through bore 40 for placing over and sealing against an exterior surface of a syringe barrel 42. The through bore 40 is sized to receive and seal against the exterior surface of the syringe barrel 42 and the plunger 34 abuts against a proximal flange 44 on the syringe barrel. Accordingly, the first barrel plunger 34 comprises two sealing surfaces, which include an interior static seal against the outer surface of the syringe barrel 42 and an outer dynamic seal against the interior surface 38 of the proximal housing.

In a similar manner, the second barrel plunger 36 is configured to seal against an exterior surface of the syringe barrel 42 and an interior wall surface 46 of the distal housing 18. Additionally, the second barrel plunger 36 incorporates a shoulder 46 for sealing against the distal opening or open end 28 of the proximal housing 20. Thus, in the embodiment shown, the second barrel plunger 36 has three sealing surfaces, two of which are static and the third is a dynamic seal against the interior wall surface 46 of the distal housing 18.

Still referring to FIG. 3, in one embodiment, the distal housing 18 has an open proximal end 48 and a closed-off distal end 50 comprising a nose section 52, which in one embodiment is an elongated cylindrical member. A third barrel plunger 54 is disposed in the distal housing 18. In one embodiment, the third barrel plunger 54 rests against and contacts the distal shoulder 56 of the distal housing. The plunger 54 is sized so that it is in static sealing arrangement with the interior wall surface 46 of the distal housing. The plunger 54 also has a central bore sized to provide a dynamic seal against the exterior wall surface of the syringe barrel 42. As further discussed below, the syringe barrel 42 may slide axially relative to the third barrel plunger 54.

The syringe 58, which includes the syringe barrel 42 and a piston rod 59, has its injection end projecting through the bore of the third plunger 54 so that the needle 60 lies coaxially with the elongated nose section 52. As further discussed below, the proximal housing 20 and the entire syringe 58, including the piston rod 59, move axially forward, in the distal direction, to puncture a subject and then the syringe barrel 42 is moved axially backward, in the proximal direction, relative to the plunger rod 59 to dispense medication housed inside the syringe barrel to the space under the skin. Thus, in accordance with one aspect of the present invention, a syringe assembly is provided wherein an injection is performed by holding a plunger rod relatively fixed while moving a syringe barrel relative to the plunger rod to perform an injection.

In an alternative embodiment, the proximal housing 20 and the entire syringe 58 move axially forward to puncture the skin. Then by placing the first barrel plunger 34 on the plunger rod 59 so that the plunger 34 is in static sealing arrangement with the plunger rod but in dynamic sealing arrangement with the interior surface 38 of the proximal housing 20, an injection may be performed in a normal fashion, i.e., by pressing on the plunger rod relative to the syringe barrel to provide an injection. In the present embodiment, the plunger rod may be moved by creating a vacuum in the space between the first barrel plunger 34 and the second barrel plunger 36.

Again referring to FIGS. 1 and 3, a gas manifold 62 is provided on the distal housing 18. In one embodiment, the gas manifold 62 comprises a gas nozzle 64, which in one embodiment is a nozzle comprising barb fitting, and a first chamber nozzle 66 and a second chamber nozzle 68. Each chamber nozzle 66, 68 is connected to a tubing 32, which is separately connected to the telescoping device 16. In one embodiment, the manifold comprises two angled needle valves 70 connected to a common inlet block 72 (FIG. 1), which has the gas nozzle 64 positioned thereon. In one preferred embodiment, a vacuum source (not shown) is connected to the gas nozzle 64. A vacuum is then directed to needle valve that is opened, by using the valve handle 74 to turn the valve open or closed. The manifold is presently contemplated to be automated by connecting the two hoses 32 to a controlled manifold. Then using a controller, a vacuum can be applied to create a vacuum in either or both hoses at the same time to perform an injection, as further discussed below. The automated header will eliminate the need to have separate valve handles to open and/or closed the valves.

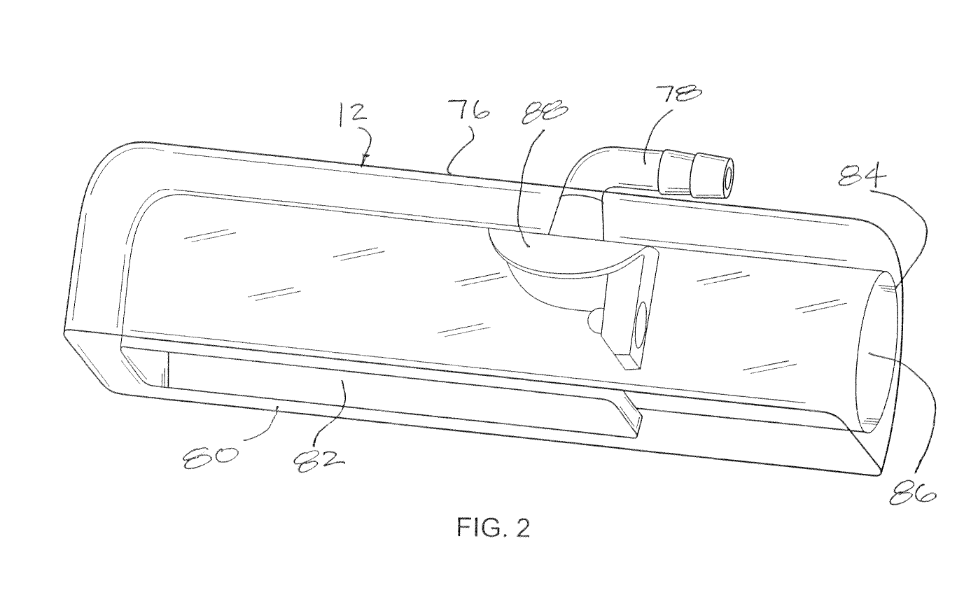

FIG. 2 is a transparent perspective view of an injection head 12 provided in accordance with aspects of the present invention. The injection head 12 includes a housing 76 and a connection nozzle 78. The housing 76 is somewhat elongated and comprises an oblong opening or stretched opening 80 that leads to an interior cavity or chamber 82 of the housing. The housing 76 further incorporates an axial opening 84, which is at an end of an axial bore 86, which is in communication with the interior cavity 82. The connection 78 may be a hose barb connector and can attach to the housing using known methods in the art, such gluing, bonding, or a threaded connector. The idea is to permit a vacuum source to be applied to the nozzle 78 to then create a vacuum in the interior cavity 82 of the housing, as further discussed below. Vacuum is applied to the telescoping device 16 by way of the communication hole 30 on the proximal housing and through a bore on the shoulder 56 of the distal housing. The bore is also provided through the third barrel plunger 54 so that a vacuum may be created in the space between the second barrel plunger 36 and the third barrel plunger 54, as further discussed below.

In one embodiment, a needle guide member 88 is located in the interior cavity 82 of the housing 76 of the injection head 12. The guide member 88 has an opening sized to receive a needle and is coaxially disposed with the bore 86. During an injection, the needle is moved axially forward through the bore 86 and the guide member 88 while vacuum is applied to draw up the skin into the cavity. As the skin is disposed in front of the needle, a puncture is made through the skin, as further discussed below.

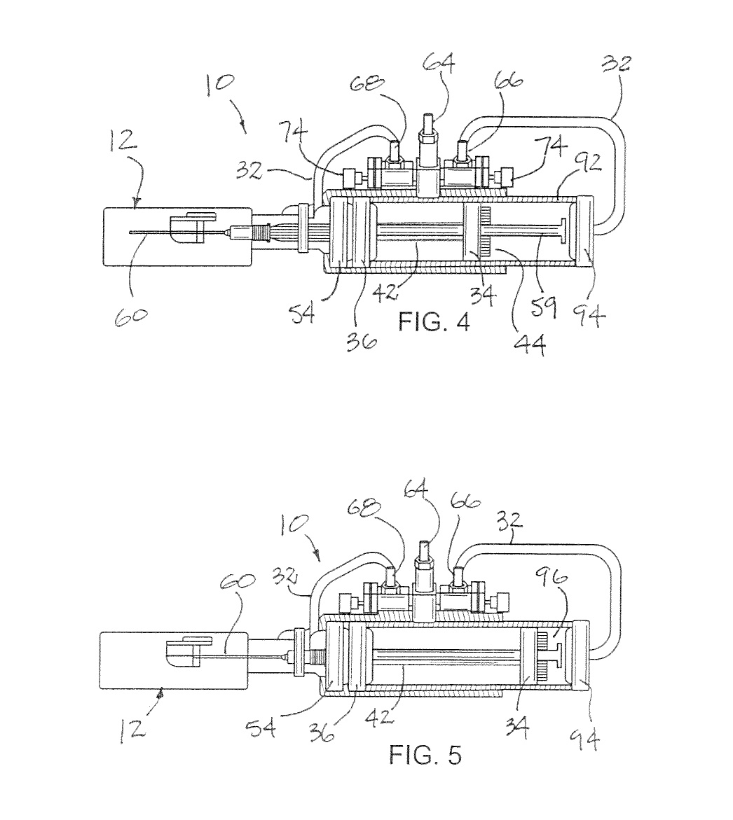

With reference now to FIGS. 4 and 5 in addition to FIGS. 1 and 3, the manner in which the needle assembly 10 may operate is discussed. Starting with FIG. 1, the needle assembly 10 is shown in a ready to use position. The nozzle 64 on the manifold 62 is connected to a vacuum source, which may be between 100 kPa (about 760 Torr) to 100 nPa (about 1×10⁻⁹ Torr), with about 3 kPa to 100 mPa (25 to 1×10⁻³ Torr) being more preferred. The nozzle 78 of the injection head 12 is also tied or connected to a vacuum source.

The injection head 12, more particularly the opening 80 of the housing of the injection head, is then placed against the skin in the general area to be injected. Vacuum is then applied to create a vacuum in the interior cavity of the injection head to pull up the skin. The valve handle 74 on the first needle valve is activated or manipulated to open the valve and create a vacuum through the second chamber nozzle 68, which creates a vacuum in the space 90 between the second barrel plunger 36 and the third barrel plunger 54. Vacuum provided to the space 90 causes the proximal housing 20 to move distally forward relative to the distal housing 18 and the two housing chambers in telescoping fashion. The needle 60, which is connected to the syringe barrel, which is mounted to the proximal housing, also moves axially forward. As further discussed below, this causes the needle to penetrate the skin that has been pulled into the cavity of the injection head 12. Accordingly, aspects of the present invention is a device for injecting the skin by moving the needle generally parallel to the surface of the skin near the injection site. At the injection site, where the skin is pulled into the injection head, the needle penetrates the skin in a perpendicular manner, as is common in the art.

FIG. 4 shows the syringe assembly 10 after vacuum is applied to the space 90 between the second plunger 36 and the third plunger 54 to move the two together. FIG. 4 (as well as FIG. 5) also shows an alternative proximal housing 92, which uses a fourth plunger 94 at the proximal end of the proximal housing 92 instead of a singularly formed proximal end, as shown in FIGS. 1 and 3. In practice, the needle 60 has now penetrated the skin following this step.

To deposit fluids in the skin, the plunger rod 59 is held steady while the syringe barrel 42 moves proximally relative to the plunger rod. In one embodiment, this is performed by opening the valve handle 74 on the needle valve to provide a vacuum through the first chamber nozzle 66. This in turn creates a vacuum in the space 96 located, between the first plunger 34 and the fourth plunger 94 (or the proximal end of the proximal housing in the first housing of FIG. 1). With reference now to FIG. 5, this causes the space 96 to collapse, or in practice the first barrel plunger 34 to move proximally towards the fourth barrel plunger 94. As the first and fourth plungers move together, the piston rod 59 is pushed further into the interior cavity of the syringe barrel to then discharge fluids out the needle. Thus, in the present embodiment, an injection is made while the syringe barrel 42 moves away from the skin, or while the needle is being retracted from the skin.

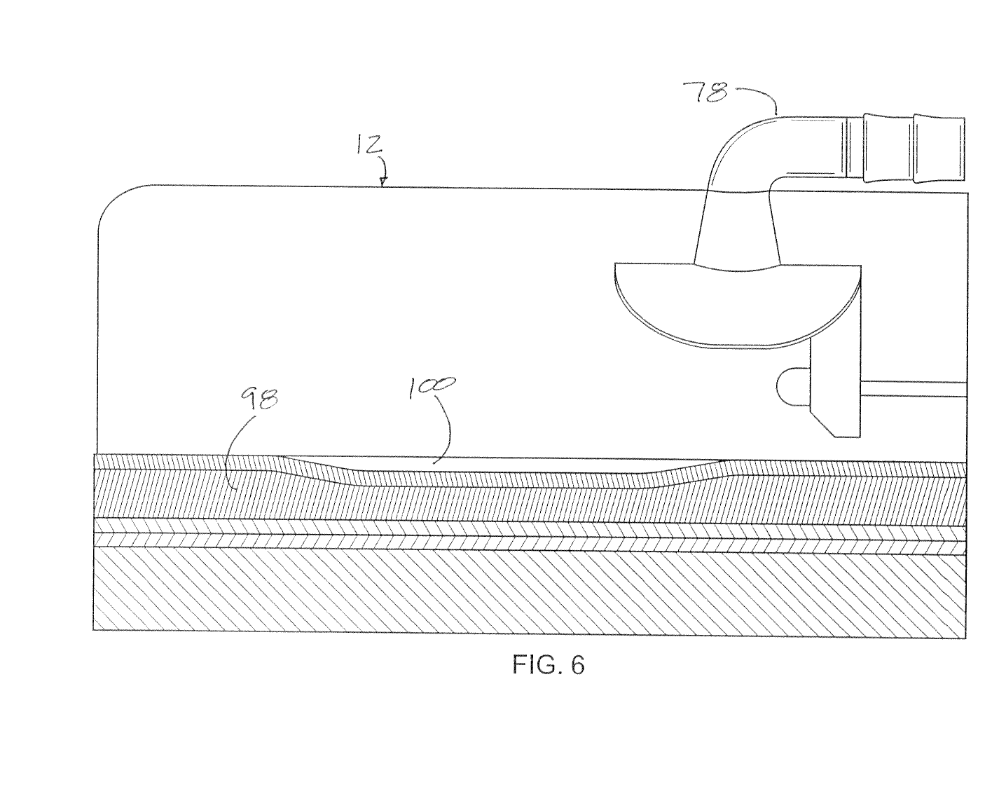

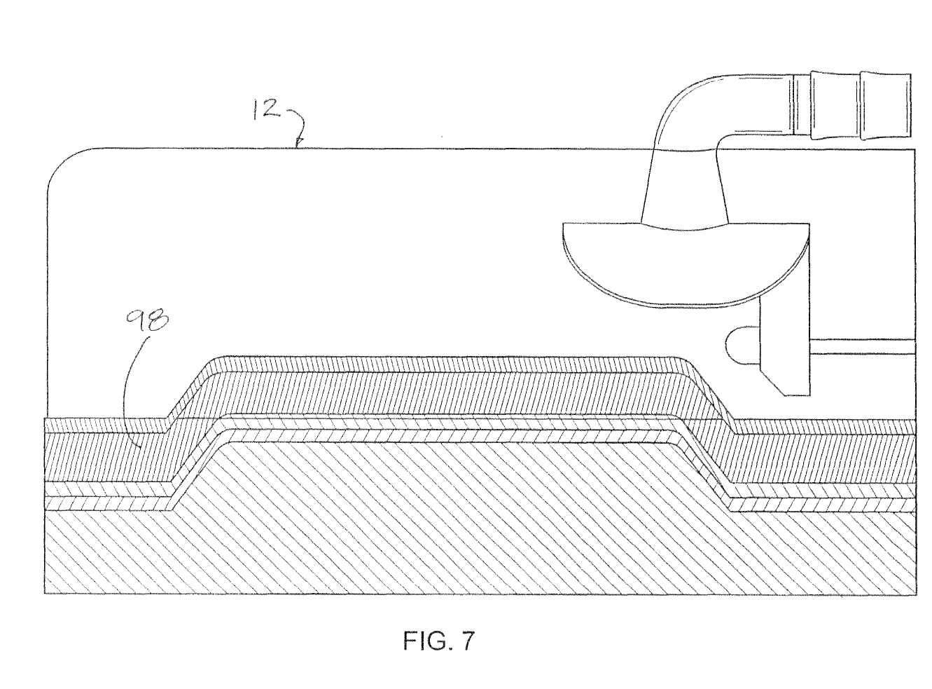

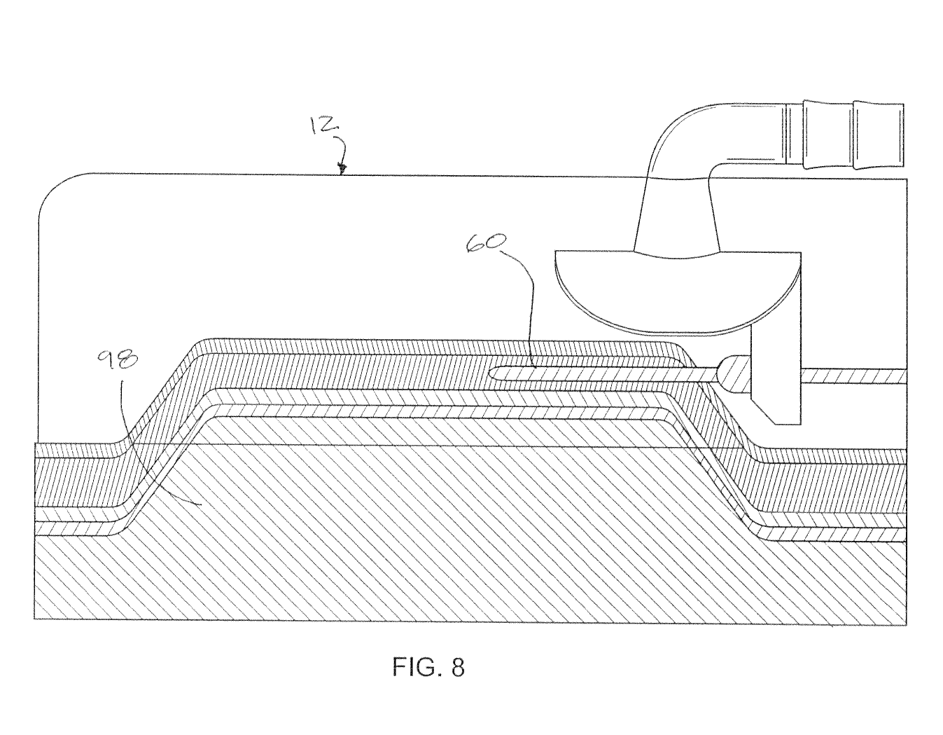

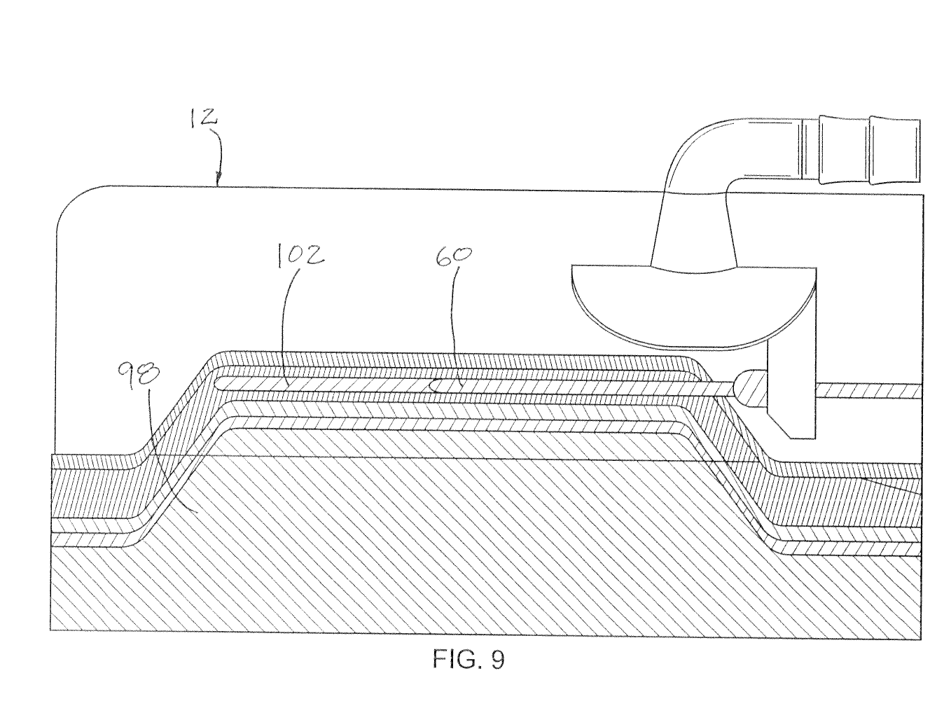

Referring now to FIGS. 6-9 for discussions on pulling up on the skin inside the injection head 12 to perform an injection. FIG. 6 is a partial cross-sectional side view of the injection head 12 placed over the skin 98 and the various skin layers. A gap 100 is shown between the housing and the skin, which represents a wrinkle on the skin. FIG. 7 shows a vacuum being applied to the housing, which then sucks the skin partially into the cavity of the housing. FIG. 8 shows the syringe device being activated to move the needle 60 into the skin to perform an injection, as discussed above.

FIG. 9 shows the needle 60 being withdrawn from the skin 98 (i.e., moved to the right of FIG. 9) while fluid 102 is concurrently deposited in the space vacated by the needle. Alternatively, the needle can be stationary to completely perform an injection and only to be removed upon completion of a pre-determined dosage, as discussed above.

Advantageously, the injection device provided herein allows a user to perform an injection that is generally parallel to the surface of the skin by pulling up on the skin, as shown in FIGS. 7-9. Broadly speaking, the injection device for performing an injection provided herein comprises a pump end for pushing a rod to perform an injection; an injection head coupled to the pump end, said injection head comprising a housing defining a cavity and having a perimeter defining an opening along a first plane: a vacuum source coupled to the injection head for providing a vacuum in the cavity of the housing; and a needle moveable along a second plane, which is non-perpendicular to the first plane.

Although limited embodiments of injection devices and their components have been specifically described and illustrated herein, many modifications and variations will be apparent to those skilled in the art. For example, while the needle is described as moving generally along a parallel plane with the opening surface of the injection head, the movement can slight converge. Accordingly, it is to be understood that the injection devices and their components constructed according to principles of this invention may be embodied other than as specifically described herein. The invention is also defined in the following claims.

What is claimed is:

1. An injection device for performing an injection comprising:
a pump end for pushing a needle to perform an injection;
an injection head coupled to the pump end, said injection head comprising a housing defining a cavity and having a perimeter defining an opening along a first plane;
a vacuum source coupled to the injection head for providing a vacuum in the cavity of the housing; and
wherein the needle is moveable along a second plane, which is non-perpendicular to the first plane.

2. An injection device for performing an injection comprising:
a pump end for pushing a needle mounted on a syringe to perform an injection;
an injection head connected to the pump end; said injection head comprising a nozzle and an opening defining a plane for placing against the skin;
a telescoping end having the syringe placed therein; said telescoping end comprising a proximal housing and a distal housing;
wherein the proximal housing and the distal housing are configured to telescopically move relative to one another when a vacuum source is applied.

3. A injection device for performing an injection comprising:
a pump end for pushing a needle mounted on a syringe to perform an injection;
an injection head connected to the pump end; said injection head comprising a nozzle and an opening defining a plane for placing against the skin;
a telescoping end having the syringe placed therein; said telescoping end comprising a proximal housing and a distal housing;
a manifold connected to the telescoping end comprising at least one valve having a valve handle.

4. The injection device of claim 3, wherein the valve is an angled needle valve.

5. The injection device of claim 3, further comprising a plurality of plungers.

6. The injection device of claim 5, wherein the syringe comprising a plunger rod comprising a plunger.

7. The injection device of claim 3, further comprising a tubing for providing communication between manifold and an interior cavity of the telescoping end.
